(19) United States
(12) Patent Application Publication
(10) Pub. No.: US 2013/0095186 A1
(43) Pub. Date: Apr. 18, 2013

(54) TEMPERATURE-SENSITIVE CARRIER FOR CARRYING A PHYSIOLOGICALLY ACTIVE SUBSTANCE AND PREPARATION METHOD THEREOF

(71) Applicants: Kun Na, Bucheon (KR); Young Seok Jung, Bucheon (KR)

(72) Inventors: Kun Na, Bucheon (KR); Young Seok Jung, Bucheon (KR)

(21) Appl. No.: 13/319,306

(22) PCT Filed: Jun. 29, 2011
(86) PCT No.: PCT/KR11/04732

§ 371 Date: Nov. 7, 2011

(30) Foreign Application Priority Data
Jun. 29, 2010 (KR) 10-2010-0061870

(51) Int. Cl. A61K 9/48; A61K 38/47

(52) U.S. Cl. CPC A61K 9/4816; A61K 38/47
USPC 424/493; 525/54.2; 424/94.61; 435/188

(57) ABSTRACT

The present invention relates to a temperature-sensitive carrier for carrying a physiologically active substance and a preparation method thereof. Specifically, the temperature-sensitive carrier according to the present invention comprises an amphiphilic biodegradable block copolymer containing polysaccharide or polysaccharide and succinic anhydride as a hydrophilic block and polylactide as a non-ionic block. A hydrophilic polymer-polylactide copolymer according to the present invention forms a stable complex with a physiologically active substance such as protein, polynucleotide and the like in vivo via ionic bonding and temperature-sensitive hydrophobic bonding. Therefore, a copolymer according to the present invention can facilitate in vivo delivery of a physiologically active substance and used as an in vivo drug delivery system.

### CROSS-REFERENCE TO RELATED APPLICATIONS

This application is a National Phase application, filed under 35 U.S.C. §371, of PCT Application No. PCT/KR2011/004732, filed Jun. 29, 2011, which claims the benefit of priority to Korean Patent Application No. 10-2010-0061870, filed on Jun. 29, 2010, the contents of which are incorporated herein by reference in their entirety.

### BACKGROUND

1. Field of the Invention

The present invention relates to a temperature-sensitive carrier for carrying a physiologically active substance and a preparation method thereof. Specifically, the temperature-sensitive carrier according to the present invention comprises an amphiphilic biodegradable block copolymer containing a polysaccharide or a polysaccharide and succinic anhydride as a hydrophilic block and polylactide as a non-ionic block.

2. Description of the Related Art

Recently, nano-structured materials have received much attention as a potentially available drug carrier. Therefore, various amphiphilic polymers comprising both a hydrophobic block and a hydrophilic block have been synthesized in order to develop more effective nano-structured materials. Since the hydrophobic blocks of such amphiphilic polymers have a tendency to self-assemble in an aqueous solution, avoiding contact with water to minimize the free energy of the system, the amphiphilic polymers form nanoparticles. At the same time, the hydrophilic blocks thereof are uniformly dissolved in an aqueous solution so that the nanoparticles can maintain a thermodynamically stable structure.

Meanwhile, researches have been in progress on the delivery of a drug by using an ioncomplex of a physiologically active substance (e.g., therapeutic proteins and genes) that possesses an electric charge in vivo with a charged polymeric material. K. Kataoka et al. proposed a novel concept of “polyion complex (PIC) micelles” that nanoparticles are formed via ionic bonding between polymers having counter ions, by using both a poly(ethylene oxide)-poly(L-lysine) block copolymer and a poly(ethylene oxide)-poly(L-aspartate) block copolymer (see A. Harada and K. Kataoka, Macromolecules, 28, 5294 (1995)). By using this concept, they reported that lysozyme, which is a positively charged protein having an isoelectric point of about 11, is successfully loaded within polymer micelles (see A. Harada and K. Kataoka, Macromolecules, 31, 288 (1998)).

Further, Biomaterials 28 (2007), pp. 4132-4142 describes a method of in vivo delivering a negatively charged drug such as a nucleotide with positively charged micelles, which are formed in an aqueous solution by using a copolymer of polyethyleneimine and polycaprolactone.

Despite continued research on an ioncomplex for drug delivery, conventional ioncomplexes still have a stability problem due to strong ion strength in the body.

In order to solve the above problems, therefore, the present inventors have endeavored to develop a polymeric material capable of employing both ionic bonding and hydrophobic bonding.

Meanwhile, the methods of encapsulating a drug may be largely divided into those using a dialysis membrane and those forming a complex of a charged drug via ion bonding. In this regard, the former is recognized to provide an encapsulated drug having a higher in vivo stability than the latter.

In the dialysis method, however, a drug dissolved in an organic solvent is incorporated into an encapsulating body by the replacement between water and the organic solvent, i.e., by the change of the system due to self-association of encapsulating body in order to decrease free energy of the system. Thus, this method is not suitable for such susceptible substances that are degenerated or degraded in an organic solvent, for example, protein.

Under the circumstances, the present inventors noted temperature sensitivity as a means of inducing the change of system for spontaneously incorporating a drug into an encapsulating body as the encapsulating body naturally decreases its free energy, and consequently have prepared a polyionic nano-complex by connecting a charged group for combining with protein and a residue for producing temperature sensitivity.

### SUMMARY

It is, therefore, an object of the present invention to provide a temperature-sensitive carrier for carrying a physiologically active substance comprising a copolymer of a combination of a polysaccharide and succinic anhydride and polylactide.

Another object of the present invention is to provide a preparation method of the above temperature-sensitive carrier for carrying a physiologically active substance.

Still another object of the present invention is to provide a pharmaceutical composition for sustained-release of a substance comprising the above temperature-sensitive carrier and a physiologically active substance encapsulated within the carrier.

In order to accomplish the above objects, there is provided a temperature-sensitive carrier for carrying a physiologically active substance comprising a copolymer of a combination of a polysaccharide and succinic anhydride and polylactide.

According to one embodiment of the present invention, the polysaccharide may be inherently charged and comprise a non-toxic unit having a molecular weight of at least 5,000.

According to one embodiment of the present invention, the polysaccharide may be a hydrophilic pullulan or hyaluronic acid derivative.

In accordance with one embodiment of the present invention, the combination of a polysaccharide and succinic anhydride may combine with the polylactide in the weight ratio of 1:0.5 to 1:5.

In one embodiment, the above-mentioned copolymer may combine with at least one physiologically active substance selected from the group consisting of protein, peptide, nucleotide, and small organic compounds having a hydrophobic or hydrophilic functional group.

In one embodiment, the copolymer may combine with the physiologically active substance via ionic bonding and hydrophobic bonding.

Further, the present invention provides a preparation method of a temperature-sensitive carrier for carrying a physiologically active substance, which comprises synthesizing a hydrophilic polymer by covalently binding a polysaccharide and succinic anhydride; and react the synthesized hydrophilic polymer with polylactide to provide a hydrophilic polymer-polylactide copolymer.

In one embodiment, the preparation method according to the present invention may further comprise forming a complex by adding a physiologically active substance to the hydrophilic polymer-polylactide copolymer.

In one embodiment, a polysaccharide and succinic anhydride may form covalent bonds in 4-dimethylaminopyridine(DMAP) solvent; and a hydrophilic polymer-polylactide copolymer may be synthesized via ring-opening polymerization of polylactide in the dimethylsulfoxide (DMSO) solvent by using triethylamine(TEA) as a catalyst.

In one embodiment, a physiologically active substance may be at least one selected from the group consisting of protein, peptide, nucleotide, and small organic compounds having a hydrophobic or hydrophilic functional group.

In one embodiment, a complex may be formed by adding a physiologically active substance to the above-mentioned copolymer in the temperature range of 4 to 10° C. below the temperature at which the copolymer exhibits temperature-sensitivity.

Further, the present invention provides a pharmaceutical composition for sustained-release of a substance comprising a temperature-sensitive carrier for carrying a physiologically active substance according to the present invention and a physiologically active substance encapsulated within the carrier.

It has been confirmed that a hydrophilic polymer-polylactide copolymer according to the present invention combines with a physiologically active substance such as protein, polynucleotide, etc. via ionic bonding and temperature-sensitive hydrophobic bonding. Accordingly, a temperature-sensitive carrier comprising a copolymer according to the present invention forms a stable complex in vivo to facilitate in vivo delivery of a physiologically active substance, and, thus, is useful as an in vivo drug delivery system.

### BRIEF DESCRIPTION OF THE DRAWINGS

FIG. 1 presents ¹H-NMR Spectrum for a polysaccharide-succinic anhydride polymer synthesized in accordance with one embodiment of the present invention.

FIG. 2 presents ¹H-NMR Spectrum for a polysaccharide-succinic anhydride-polylactide copolymer synthesized in accordance with one embodiment of the present invention.

FIG. 3 shows transmittance variations (% T) depending on the polymerization ratios of polysaccharide-succinic anhydride-polylactide copolymers synthesized in accordance with one embodiment of the present invention and a temperature change.

FIG. 4 is a graph showing a temperature-dependent particle diameter distribution of a complex of a polysaccharide-succinic anhydride-polylactide copolymer synthesized in accordance with one embodiment of the present invention and a physiologically active substance.

FIG. 5 is a result of analyzing the formation of a complex by attaching fluorescent labels to a polysaccharide-succinic anhydride-polylactide copolymer synthesized in accordance with one embodiment of the present invention and a physiologically active substance and measuring the fluorescence intensity thereof.

FIG. 6 is a result of analyzing the degradation of a complex in vitro by attaching fluorescent labels to a polysaccharide-succinic anhydride-polylactide copolymer synthesized in accordance with one embodiment of the present invention and a physiologically active substance.

FIG. 7 is a result of analyzing the degradation of a complex in vivo by attaching fluorescent labels to polysaccharide-succinic anhydride-polylactide polymer synthesized in accordance with one embodiment of the present invention and a physiologically active substance.

### DETAILED DESCRIPTION OF EXEMPLARY EMBODIMENTS

The present invention is characterized by providing a biocompatible polymer having amphiphilicity and temperature-sensitivity, which can be used as a drug delivery system, etc. Specifically, the present invention is characterized by providing an amphiphilic, biodegradable and temperature-sensitive carrier for carrying a physiologically active substance, which comprises a copolymer having a hydrophilic polymer of a polysaccharide and succinic anhydride as a hydrophilic block and polylactide as a non-ionic block.

Especially, the temperature-sensitivity of a carrier according to the present invention can be adjusted by the polymerization ratios of polylactide used as a non-ionic block in the copolymer, thereby modifying the phase transition temperature of the copolymer.

Further, a polysaccharide-succinic anhydride polymer, etc. may be used as an initiator. A hydrophilic polymer of a polysaccharide and succinic anhydride may combine with polylactide having temperature-sensitivity and hydrophobicity via ring-opening polymerization to provide a polysaccharide-succinic anhydride-polylactide copolymer. The hydrophobicity of the copolymer can increase as the polymerization ratio of the non-ionic polymer, i.e., polylactide increases.

Accordingly, the formation and drug release behavior of a carrier according to the present invention can be reversibly modified by adjusting the polymerization ratio of polylactide used for the formation of the carrier and a temperature. In other words, since synthesis of a carrier can be controlled by adjusting a temperature and the polymerization ratio of polylactide, the carrier is useful as a drug delivery system capable of easily encapsulating a drug and controlling release of the drug.

Further, a temperature-sensitive carrier according to the present invention is characterized by employing a polysaccharide-succinic anhydride polymer as a hydrophilic polymer by adopting a polysaccharide as a biodegradable polymer for the purpose of securing in vivo safety and by adding succinic anhydride to the polysaccharide for the purpose of carrying ionicity.

Meanwhile, in conventional methods for preparing a nano-level complex, an amphiphilic polymer forms a micelle by replacement of solvent through a dialysis membrane, or a complex is formed by spreading a polymer into a film at an elevated temperature in the reactor. Therefore, such methods cannot be readily applied to drugs that are unstable in an organic solvent or at an elevated temperature.

As a means for solving the above problems, the present invention employs succinic anhydride to endow a polymeric material with ionicity, thereby allowing a physiologically active substance to readily form a complex with polymeric material via ionic bonding.

The preparation method of a temperature-sensitive carrier according to the present invention will be described in detail hereinbelow.

### Step 1: Covalently Binding a Polysaccharide and Succinic Anhydride

In order to prepare a temperature-sensitive earlier according to the present invention, first, polysaccharide and succinic anhydride are combined with each other via covalent bonding. A polysaccharide useful in the present invention should have superior to biocompatibility, biodegradability and in vivo stability. Any biocompatible polysaccharide or polysaccharide derivative, for example, an inherently charged polysaccharide or a polysaccharide combined with a charged material can be used as a polysaccharide of the present invention. Preferably, a hydrophilic pullulan or hyaluronic acid derivative can be used.

In one embodiment, pullulan is used as a polysaccharide of the present invention. The plullan is obtained by isolating and purifying a polysaccharide produced by Aureobasidium pullulans (DE BARY) ARN, and the main component thereof is neutral polysaccharides. Polysaccharides are well dissolved in water but not dissolved in alcohols and oils. They are stable to acid, alkali, heat, etc. although having a relatively low viscosity as compared with other gums. Especially, they have strong adhesive force together with encapsulating capability, two kinds of average molecular weight (i.e., 200,000 and 100,000), and a viscosity of 12 cps. A polysaccharide of the present invention may comprise a non-toxic unit having a molecular weight of at least 5,000. In one embodiment, a polysaccharide having a molecular weight of 100,000 is used.

As a polysaccharide according to the present invention, commercially available ones or polysaccharides that are isolated from nature and purified may be used. Preferably, impurities are eliminated from a polysaccharide and the polysaccharide having an increased purity may be used.

As a polysaccharide used in one embodiment, pullulan has the following structure:

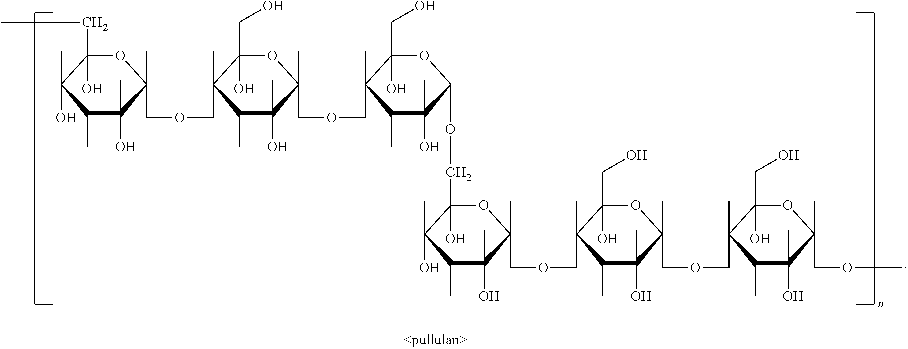

Meanwhile, in order to covalently bind a polysaccharide and succinic anhydride, a polysaccharide is dissolved in an organic solvent and then reacted with succinic anhydride to form covalent bonds. Preferably, an organic solvent is used in a sufficient amount to fully dissolve a polysaccharide. If the amount of an organic solvent is too small, the polysaccharide can adhere to each other.

An organic solvent according to the present invention is not limited to, but may be DMSO, formamide or DMF, and preferably, DMSO.

Further, succinic anhydride may be used as a charged material according to the present invention. Succinic anhydride, which combines with a polysaccharide via covalent bonding, is an anhydride of succinic acid, an organic compound having a ring structure. It has a molecular formula of C₄H₄O₃ and a molecular weight of 100.07.

In preparing a copolymer of the present invention, succinic anhydride is used for endowing a hydrophilic neutral polysaccharide of the present invention with ionicity. Specifically, succinic anhydride dissolved in DMSO is activated by DMAP and connected to a hydroxyl group of a polysaccharide. As a result, succinic anhydride provides the polysaccharide with a carboxylic group capable of exhibiting ionicity. The ionicity producing mechanism by succinic anhydride is illustrated as follows:

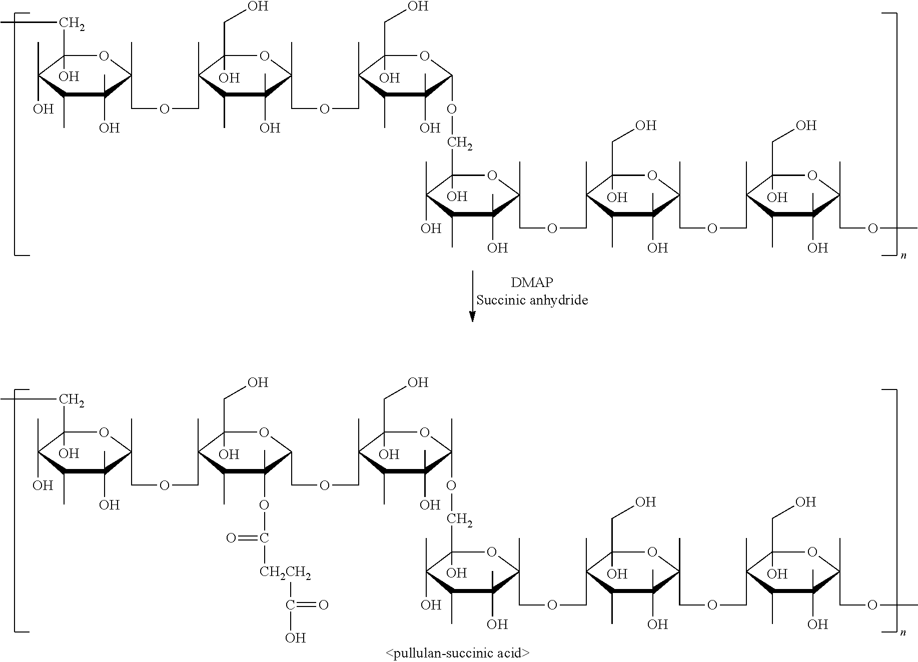

In one embodiment, pullulan as a polysaccharide is dissolved in DMSO; succinic anhydride dissolved in DMSO is activated by dimethylaminopyridine; and the activated succinic anhydride is added dropwise to, and reacted with the pullulan dissolved in DMSO to covalently bind pullulan and succinic anhydride.

### Step 2: Synthesizing a Polysaccharide-Succinic Anhydride-Polylactide Copolymer by Using a Polysaccharide-Succinic Anhydride Polymer as an Initiator

As another component of a copolymer according to the present invention, polylactide comprises a lot of methyl groups and increases the hydrophobicity of a polysaccharide to be combined due to its own non-ionic property. Consequently, it enables a copolymer to form hydrophobic bonds with a physiologically active substance to be delivered by endowing the copolymer with hydrophobicity.

Since polylactide comprises a lot of methyl groups, its degree of freedom in the aqueous milieu can be changed by a temperature change. Therefore, a temperature change can induce hydrogen bonding between polylactide and a polysaccharide and modify the hydrophobicity to be given to a copolymer.

Especially, as a temperature increases, the hydrophobicity of copolymers becomes stronger to strengthen binding force between the copolymers. Therefore, a physiologically active substance incorporated therein can be delivered to a target in a more stable manner.

As described above, once a hydrophilic polymer is synthesized by covalently binding a polysaccharide and succinic anhydride, the polymer is reacted with polylactide to obtain a hydrophilic polymer-polylactide copolymer.

In this case, polylactide can be grafted by ring-opening polymerization in which a hydroxyl group of the polysaccharide-succinic anhydride polymer serves as a multiple initiator and triethylamine(TEA) serves as a ring-opening catalyst in the DMSO solvent. Further, the hydrophobicity and temperature-sensitivity of a copolymer according to the present invention can be controlled by adjusting the amount of polylactide to be grafted. A polysaccharide-succinic anhydride polymer and polylactide can be combined in the weight ratio of 1:0.5 to 1:5.

The structure of a polysaccharide-succinic anhydride-polylactide according to the present invention and the ring-opening mechanism of polylactide are illustrated as follows:

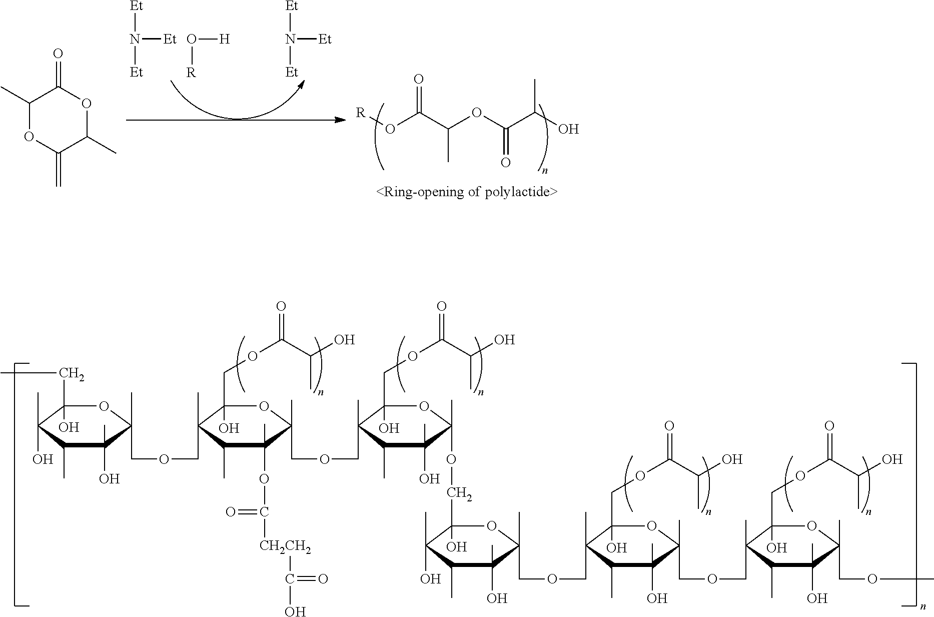

<A Polysaccharide-Succinic Anhydride-Polylactide Copolymer (Pullulan-S.A.-Poly-(L-Lactide))>

### Step 3: Forming a Complex of a Polysaccharide-Succinic Anhydride-Polylactide Copolymer and a Physiologically Active Substance

A complex can be formed by adding a physiologically active substance to the hydrophilic polymer-polylactide copolymer synthesized in step 2.

A physiologically active substance according to the present invention may be any substance having a desired pharmacological activity. Non-limiting examples of a physiologically active substance include proteins, peptides, nucleotides and small organic compounds having a hydrophobic or hydrophilic functional group.

In one embodiment, lysozyme from chicken egg white is used. Since lysozyme having an isoelectric point of 9.2 is negatively charged in vivo (pH 7.4), it can be combined with a copolymer synthesized in the present invention via ionic bonding. Ionic bonding and hydrophobic bonding induced by temperature-sensitivity are involved in the formation of a complex between a physiologically active substance and a copolymer in accordance with the present invention. The strength of hydrophobic bonding involved in the formation of a complex is determined by a temperature change. Therefore, since copolymers alone can form aggregates at a temperature higher than the phase transition temperature, it is preferred that a copolymer form a complex with a physiologically active substance via ionic bonding under the refrigerating conditions of 4° C. to 10° C. at which hydrophobicity is minimized. Then, hydrophobic bonding can be induced by increasing a temperature.

After completing the formation of a complex in Step 3, a copolymer according to the present invention and a physiologically active substance can be labeled by fluorescent labeling materials in order to measure in vivo release of the physiologically active substance and in vivo stability of a complex.

Since a complex according to the present invention is basically based on ionic bonding, the complex can be degraded by salts and serum in the body, and, in this case, the complex would lose its function and a physiologically active substance would become exposed to in vivo environment.

In one embodiment, in order to measure in vivo stability of a complex according to the present invention, Cy5.5 (Amersham, SWE) is attached to a physiologically active substance, and BHQ-3 (biosearch technologies, USA.), which specifically quenches the fluorescence of Cy5.5, is attached to a copolymer according to the present invention. The excitation wavelengths of physiologically active substances alone and in combination with a copolymer (i.e., a complex) are fixed to 675 nm; and the stability of the complex is determined by using the difference in fluorescence intensity between at the excitation wavelength of 675 nm and at emission wavelength of 695 nm.

As a result, it is confirmed that a physiologically active substance in the form of a complex remains in the body more stably for a longer time than a physiologically active substance alone. In other words, a complex containing polylactide can keep a physiologically active substance more stably for a longer time and then gradually release the substance when compared with a complex not containing polylactide (see Experimental Example 4).

Therefore, a temperature-sensitive carrier according to the present invention allows a physiologically active substance incorporated therein to be retained in the body for a longer time.

As described above, the present invention can provide a method for preparing a temperature-sensitive carrier for carrying a physiologically active substance and a temperature-sensitive carrier manufactured by the method.

A carrier according to the present invention, which is prepared by a method as described above, can stably exist as a nanoparticle, e.g., a nanogel or a nano-microsphere, in aqueous milieu. Its average particle size is in the range of 100 to 200 nm.

Further, the present invention provides a composition for sustained-release of a substance, which comprises a temperature-sensitive carrier for carrying a physiologically active substance and a physiologically active substance encapsulated therein.

Thanks to the sustained-release property, a composition according to the present invention can be useful for delivering into the body protein or peptide drugs that have very short in vivo half lives but need to remain in the body for a prolonged time to produce a therapeutic effect (e.g., TRAIL, VEGF, bFGF). Especially, because a carrier according to the present invention has stronger hydrophobicity in vivo than in vitro due to its temperature-sensitivity, its sustained-release property is much superior to that of other carriers employing ionic bonding. Thus, a carrier according to the present invention is suitable for providing a pharmacological composition for releasing a drug for a prolonged time.

Also, a composition according to the present invention can be used as a complex with an anti-cancer agent for targeting the anti-cancer agent into cancer cells based on an EPR effect resulting from the size of a nano-microsphere.

Unless defined otherwise, the term “treating,” as used herein, refers to reversing, alleviating, inhibiting the progress of, or preventing the disorder or condition to which such term applies, or one or more symptoms of such disorder or condition. The term “treatment,” as used herein, refers to the act of treating, as “treating” is defined immediately above.

Preferably, a temperature-sensitive carrier for carrying a physiologically active substance according to the present invention may be used for treating cancer.

A pharmacological composition according to the present invention may contain a pharmacologically effective amount of a carrier according to the present invention alone or in combination with at least one pharmacologically acceptable vehicle, excipient or diluent. The term “pharmacologically effective amount,” as used herein, refers to an amount of a carrier sufficient to prevent, improve or treat target diseases.

In the pharmacological composition according to the present invention, a pharmacologically effective amount of a carrier according to the present invention may be in the range of 0.5 to 100 mg/day/kg body weight, preferably, 0.5 to 5 mg/day/kg body weight. However, the pharmacologically effective amount can vary with the kind and the severity of the disease to be treated, age, body weight and the physical condition of the patient to be treated, administration route, duration of therapy and the like.

The term “pharmacologically acceptable” refers to molecular entities and compositions that are physiologically tolerable and do not typically produce an allergic or similar untoward reaction, such as gastric upset, dizziness and the like, when administered to a human. Non-limiting examples of a vehicle, an excipient and diluent are lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxy benzoate, propyl hydroxy benzoate, talc, magnesium stearate, mineral oil and the like. Further, fillets, anticoagulants, lubricants, wetting agents, flavoring agents, emulsifier, preservatives, etc. can be further contained in the composition of the present invention.

A pharmacological composition according to the present invention may be formulated into a suitable formulation in accordance with the methods known to those skilled in the art so that it can provide a controlled or sustained release of a substance after being administered into a mammal. Non-limiting examples of the formulation are powder, granules, tablets, emulsions, syrups, aerosols, soft or hard gelatin capsules, sterilized injection solution, sterilized powder and the like.

A pharmacological composition according to the present invention may be administered by various routes including oral, transdermal, subcutaneous, intravenous or intramuscular administration. The amount of an active ingredient to be administered can be adjusted based on an administration route, age, sex, body weight of a patient, the severity of a disease, etc.

Meanwhile, since a carrier according to the present invention contains a hydrophilic polymer and a hydrophobic polylactide, it can form a nano-microsphere, which can further contain a therapeutically active agent or a biological agent, preferably, anticancer agent. In this case, the agent may be encapsulated into the nano-microsphere.

The present invention will now be described in more detail with reference to the following examples. However, these examples are given by way of illustration only not of limitation.

### Example 1

### Preparation of a Polysaccharide-Succinic Anhydride-Polylactide Copolymer

### <1-1> Covalently Binding a Polysaccharide and Succinic Anhydride

In a 100 ml flask, 600 mg of sucinnic anhydride (sigma, 100.07 da) was dissolved in 10 ml of dimethylsulfoxide (DMSO, sigma) and reacted with 4-dimethylaminopyridine (DMAP) for 8 hours. Then, 5 g of pullulan was dissolved in 50 ml of DMSO and the above activated succinic anhydride was added dropwise thereto. After 24 hours of the reaction, the organic solvent, the unreacted material and byproducts were removed from the reaction mixture by using a conventional dialysis method. A pullulan-succinic anhydride polymer was obtained by using a freeze dryer.

### <1-2> Synthesizing a Pollulan-Succinic Anhydride-Polylactide Copolymer

0.5 g of the pullulan-succinic anhydride polymer obtained in Step <1-1> above was reacted with 0.9 to 1.3 g of polylactide in 30 ml of dimethylsulfoxide (DMSO, sigma) by using triethylamine(TEA, sigma) as a ring-opening catalyst. After 12 hours of the reaction at 75° C., the organic solvent, the unreacted material and byproducts were removed from the reaction mixture by using a dialysis membrane. A pullulan-succinic anhydride-polylactide copolymer was recovered by using a freeze dryer. The amounts of reactants and the yields are described in the following Table 1.

TABLE 1
Synthesis of a pullulan-succinic anhydride-polylactide copolymer

| | The amounts of reactants | | The amount | Solubility | |
|---|---|---|---|---|---|
| | | Poly(L- | of solvent | (in distilled | yield |
| Code | Pullulan-S.A | lactide) | (DMSO) | water) | (wt %) |
| PSPL1 | 0.5 g | 0.9 g | 30 ml | 0 | 82 |
| PSPL2 | 0.5 g | 1.1 g | 30 ml | 0 | 76 |
| PSPL3 | 0.5 g | 1.3 g | 30 ml | 0 | 80 |

### Example 2

### Formation of a Complex of a Pullulan-Succinic Anhydride-Polylactide Copolymer and a Physiologically Active Substance

In order to form a complex of a pullulan-succinic anhydride-polylactide copolymer and lysozyme (lysozyme from chicken egg white, sigma) as a physiologically active substance, 0.001 g/L of a lysozyme solution and 0.01 g/L of anionic synthetic polymer solutions comprising a pullulan-succinic anhydride-polylactide copolymer (i.e., PSPL1, PSPL2 or PSPL3 prepared in Example 1) were prepared with distilled water (pH 7.4) at 4° C. at which the hydrophobicity of the copolymer is minimized. Then, 1 ml of the lysozyme solution was added to 1 ml of each anionic synthetic polymer solution to form a complex via ionic bonding.

Meanwhile, the temperature-sensitivity of polylactide makes a copolymer more hydrophobic at an increased temperature. Further, it is difficult to form a uniform complex of a copolymer and protein at a concentration greater than the critical micelle concentration because the copolymer naturally forms an aggregate at such a concentration. For the above reasons, the PSPL1, PSPL2 or PSPL3 solution was prepared as the concentration of 0.01 g/L at 4° C. in this example. In other words, formation of a complex of a copolymer and a physiologically active substance is preferably conducted at a concentration less than the critical micelle concentration of the copolymer and at a low temperature.

### Example 3

### Labeling a Complex of a Pullulan-Succinic Anhydride-Polylactide Copolymer and a Physiologically Active Substance with Fluorescent Labels

In a 25 ml flask, 13 mg of lysozyme (sigma, 14.3 Kda) was dissolved in 14 ml of sodium carbonate buffer solution (100 mM) and 1 mg of Cy5.5 mono NHS ester(Amersham, SWE) dissolved in 0.5 ml of DMSO was added dropwise thereto. The reaction was carried out at 4° C. for 8 hours. Then, the organic solvent, the unreacted materials and byproducts were removed from the reaction mixture by using a dialysis membrane to obtain lysozyme labeled with Cy5. The material thus obtained was dispensed into 1 ml aliquots and kept in a deep freezer.

Further, 200 mg of the pullulan-succinic anhydride-polylactide copolymer synthesized in Example 1 was dissolved in 19 ml of dimethylformamide (DMF, Junsei), and BHQ-3 succinimide ester (biosearch technologies, USA) that had been dissolved in 1 ml of dimethylformamide (DMF, Junsei) was added dropwise thereto. After 8 hours of the reaction, the organic solvent, the unreacted materials and byproducts were removed from the reaction mixture by using a dialysis membrane. Finally, a pullulan-succinic anhydride-polylactide copolymer labeled with BHQ-3 was obtained by a freeze dryer.

### Experimental Example 1

### Identification of the Synthesized Pullulan-Succinic Anhydride-Polylactide Copolymer

The synthesized pullulan-succinic anhydride-polylactide copolymer was identified by using ¹H-NMR (Avancek 500, Bruker, Germany). First, in order to identify the synthesized pullulan-succinic anhydride polymer, the polymer was dissolved in D₂O and analyzed in accordance with a ¹H-NMR analysis method known in the art. Further, in order to identify the synthesized pullulan-succinic anhydride-polylactide copolymer, the copolymer was dissolved in DMSO and analyzed in the same way as above.

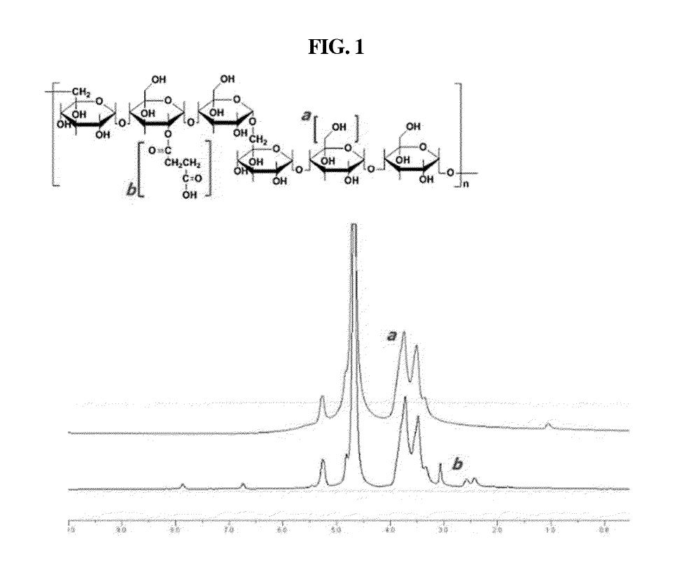

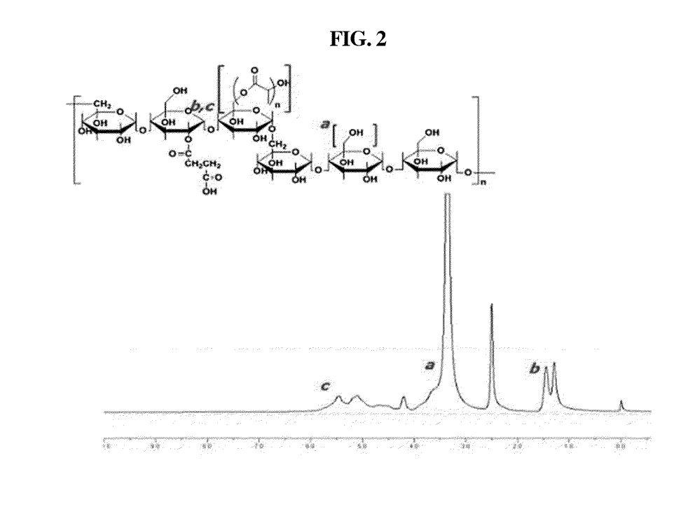

As a result, the chemical structures of the synthesized polymer and copolymer were confirmed as illustrated in FIGS. 1 and 2. The number-average molecular weight of the to polymer and copolymer and the contents of each unit, which were calculated by integrating a CH₂OH signal of pullulan (6.0˜3.0 ppm), a CH₂CH₂ signal of succinic anhydride (3.2˜3.0 ppm) and a CH₃ signal of polylactide (1.6˜10 ppm), are indicated in the following Table 2.

TABLE 2
Analysis of the contents of each unit and the
number-average molecular weight of the polymers

| | Content of each unit in 1 mol of the polymer | | | Number-average |
|---|---|---|---|---|
| Kind of | | Succinic | Poly(L- | molecular weight |
| polymer | Pullulan | anhydride | lactide) | (Mn, KDa) |
| PS | 95.3 | 4.7 | 0 | 10.5 |
| PSPL1 | 79.6 | 3.9 | 16.5 | 12.0 |
| PSPL2 | 77.4 | 3.8 | 18.8 | 12.4 |
| PSPL3 | 68.5 | 3.4 | 28.1 | 14.1 |

### Experimental Example 2

### Determination of Temperature-Sensitivity

In order to determine a difference in temperature-sensitivity of the copolymers synthesized in the above examples, transmittance in the wavelength of 500 nm was measured by using an UV spectrophotometer (UV-2450, shimadzu, Japan), while changing a temperature.

First, the pullulan-succinic anhydride-polylactide copolymers synthesized in Step <1-2> of Example 1 were dissolved in distilled water so as to be a concentration of 5 mg/ml and kept at 4° C.

Transmittance variations of the copolymers depending on a temperature change were measured at a temperature from 5° C. to 60° C., while increasing a temperature by 5° C. In order to secure the accuracy of the measurement, the interval between temperature changes was set to 30 minutes and the transmittance at each temperature was measured after stabilizing the copolymer. As a comparative group, a pullulan-succinic anhydride polymer not containing polylactide was used.

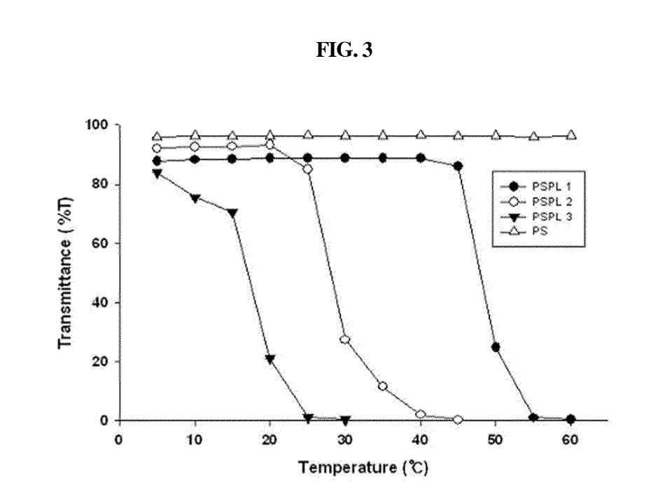

As indicated in FIG. 3 and the following Table 3, the pullulan-succinic anhydride-polylactide copolymers showed temperature-sensitivity, while the pullulan-succinic anhydride polymer not containing polylactide did not show temperature-sensitivity. Further, it was confirmed that as the content of polylactide in the copolymer increases, the copolymer exhibits temperature-sensitivity at a lower temperature.

Based on the above, the present inventors found that a copolymer showing temperature-sensitivity at a temperature the same as or lower than the body temperature of 37.5° C. (for example, PSPL1 or PSPL2) was more advantageous to form a complex with a physiologically active substance and produce temperature-sensitivity. Especially, PSPL1 was most advantageous to form a complex with a physiologically active substance since it shows temperature-sensitivity at a temperature near the body temperature of 37.5° C., thereby having a superior in vivo stability as compared with the other copolymers.

TABLE 3
Results of determining temperature-sensitivity of each
pullulan-succinic anhydride-polylactide copolymer

| | Temperature at which the copolymer |
|---|---|
| Kind of copolymer | shows temperature sensitivity (° C.) |
| PS | N.D |
| PSPL1 | 40~50 |
| PSPL2 | 25~30 |
| PSPL3 | 15~20 |

### Experimental Example 3

### Analysis of Particle Properties of a Complex Containing a Pullulan-Succinic Anhydride-Polylactide Copolymer and Lysozyme

In 1 ml of a nano-complex wherein a pullulan-succinic anhydride-polylactide to copolymer was combined with lysozyme in the ratio of 10:1, the average particle diameter was measured by using ZetasizerSZ (Malvern, UK) with a scattering angle being fixed to 90°.

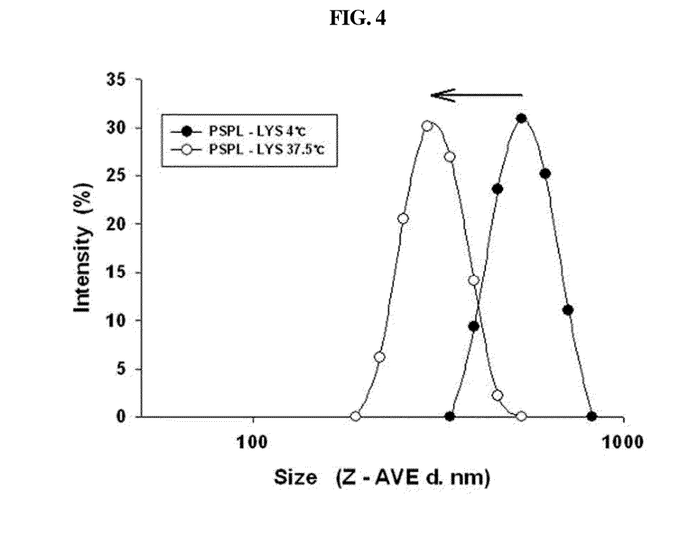

As illustrated in FIG. 4, the particle size distribution of the copolymer was shift to smaller particle sizes at the body temperature of 37.5° C. than at the refrigerating temperature of 4°. Such temperature increase greatly improves the hydrophobicity of the complex and strengthens the binding force between the copolymers, thereby allowing a physiologically active substance to be delivered in a more reliable and stable manners.

### Experimental Example 4

### Determination of In Vivo Stability of a Complex Containing a Pullulan-Succinic Anhydride-Polylactide Copolymer and a Physiologically Active Substance

In order to determine in vivo stability of a complex according to the present invention depending on salt and serum concentrations, the following experiments were conducted by using a nano-complex wherein a pullulan-succinic anhydride-polylactide copolymer labeled with BHQ-3 and lysozyme labeled with Cy5.5 were combined in the ratio of 10:1.

<4-1> In vitro analysis

The fluorescence intensity of Lysozyme labeled with Cy5.5 alone or in combination with a pullulan-succinic anhydride-polylactide copolymer labeled with BHQ-3 was determined by using RF-5301 (shimadzu). The emission wavelength was fixed to 675 nm and the fluorescence intensity was measured at the excitation wavelength of 695 nm.

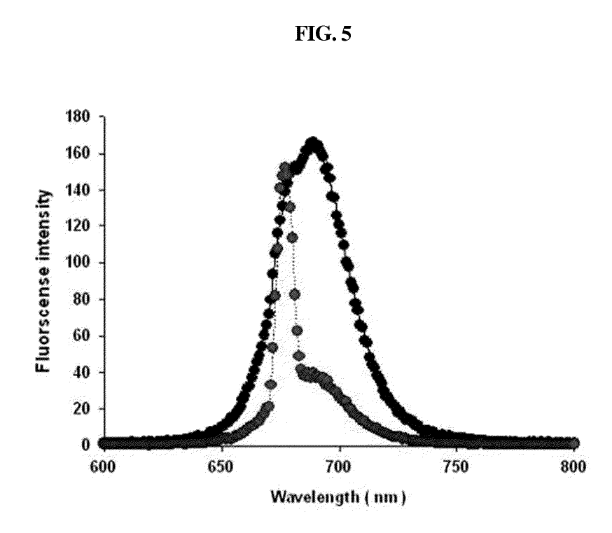

As a result, as illustrated in FIG. 5, it was found that lysozyme in combination with a pullulan-succinic anhydride-polylactide copolymer produced less fluorescence than lysozyme alone. Based on this finding, therefore, it was confirmed whether a complex remained intact or degraded.

Specifically, samples having various concentrations of salt/serum (0 mM/0%˜600 mM/40%) were prepared, and 0.2 ml of a complex was injected to 1.8 ml of each sample. Then, the stability of a complex depending on salt and serum concentrations was measured based on the change of fluorescence intensity caused by the formation of a complex.

The fluorescence intensities of lysozyme labeled with Cy5.5 alone and in combination with a pullulan-succinic anhydride-polylactide were respectively assumed to 100 and 0. The stability of a complex was calculated by substituting the fluorescence intensity value measured at the excitation wavelength of 695 nm into the following equation:

$$\frac{Sample - complex}{Lysozyme - complex} \times 100 ( \% )$$

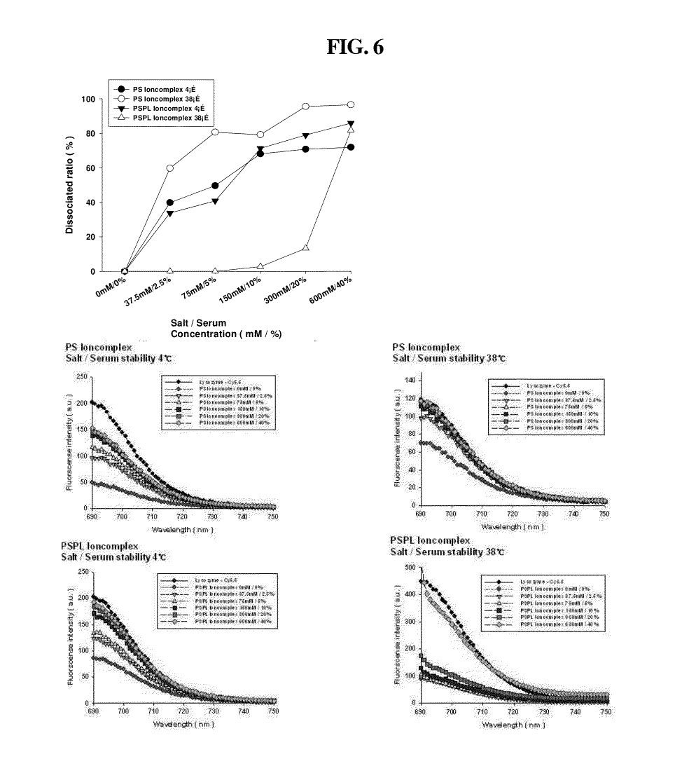

As shown in FIG. 6, in case that a pullulan-succinic anhydride polymer was not grafted with polylactide and in case that a pullulan-succinic anhydride-polylactide copolymer was not endowed with hydrophobicity resulting from a temperature increase, the complex was degraded by changing salt and serum concentrations, failing to maintain it form. On the contrary, in case that a pullulan-succinic anhydride-polylactide copolymer was endowed with hydrophobicity by increasing a temperature in accordance with the present invention, the complex stably maintained its form even at the salt and serum concentrations of in vivo environment (150 mM/10%).

<4-1> In vivo analysis

In order to confirm whether a complex according to the present invention exhibits temperature-sensitivity in vivo, a complex of a pullulan-succinic anhydride polymer and lysozyme (∘) and a complex of a pullulan-succinic anhydride-polylactide copolymer and lysozyme (□) were subcutaneously injected to nude mice (Can Cg-Foxnl-nu/CrljBgi, orient), and, then, the degree and the duration of release of lysozyme were measured.

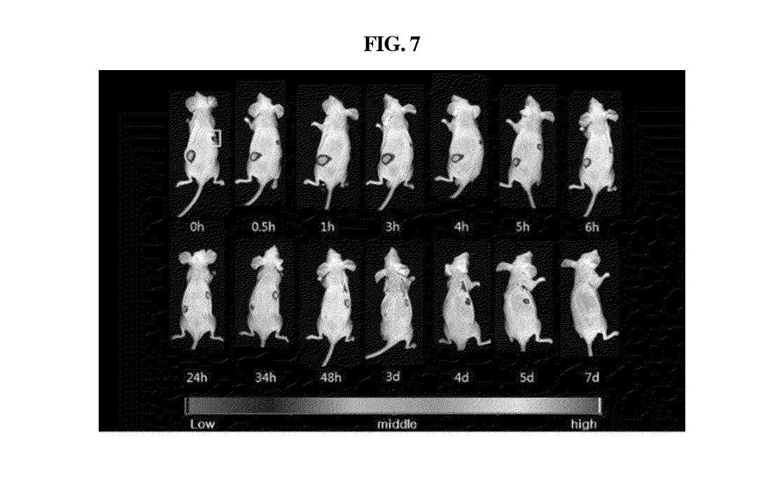

As a result, as illustrated in FIG. 7, in case of a pullulan-succinic anhydride/lysozyme complex not containing polylactide as a temperature-sensitive material, the release of lysozyme started immediately after the injection and was completed in about 24 hours. However, in case of a complex of a pullulan-succinic anhydride-polylactide copolymer and lysozyme, the release of lysozyme started about 24 hours after the injection and continued for about 7 days.

Based on the above results, it was found that a copolymer according to the present invention employing a combination of a polysaccharide and succinic anhydride as a hydrophilic block and polylactide as a non-ionic block is useful as a carrier for carrying a physiologically active substance into the body in a stable manner.

While the invention has been shown and described with respect to the preferred embodiments, it will be understood by those skilled in the art that various changes and modifications may be made without departing from the spirit and scope of the invention as defined in the following claims.

1. A temperature-sensitive carrier for carrying a physiologically active substance, which comprises a copolymer of (i) a combination of a polysaccharide and succinic anhydride and (ii) polylactide.

2. The temperature-sensitive carrier according to claim 1, wherein said polysaccharide is inherently charged and comprises a non-toxic unit having a molecular weight of at least 5,000.

3. The temperature-sensitive carrier according to claim 2, wherein said polysaccharide is a hydrophilic pullulan or hyaluronic acid derivative.

4. The temperature-sensitive carrier according to claim 1, wherein (i) a combination of a polysaccharide and succinic anhydride and (ii) polylactide are combined in the weight ratio of 1:0.5 to 1:5.

5. The temperature-sensitive carrier according to claim 1, wherein said carrier is combined with at least one physiologically active substance selected from the group consisting of protein, peptide, nucleotide and small organic compounds having a hydrophobic or hydrophilic functional group.

6. The temperature-sensitive carrier according to claim 5, wherein said physiologically active substance is combined with said copolymer via ionic bonding and hydrophobic bonding.

7. A preparation method of a temperature-sensitive carrier for carrying a physiologically active substance, which comprises:
synthesizing a hydrophilic polymer by covalently binding a polysaccharide and succinic anhydride; and
reacting the hydrophilic polymer synthesized above with polylactide to provide a hydrophilic polymer-polylactide copolymer.

8. The preparation method according to claim 7, further comprising adding a physiologically active substance to said hydrophilic polymer-polylactide copolymer to form a complex.

9. The preparation method according to claim 7, wherein said polysaccharide and said succinic anhydride form covalent bonds in 4-dimethylaminopyridine(DMAP) solvent; and said hydrophilic polymer-polylactide copolymer is synthesized via ring-opening polymerization of polylactide in the dimethylsulfoxide (DMSO) solvent by using triethylamine(TEA) as a catalyst.

10. The preparation method according to claim 8, wherein said physiologically active substance is at least one selected from the group consisting of protein, peptide, nucleotide and small organic compounds having a hydrophobic or hydrophilic functional group.

11. The preparation method according to claim 8, wherein said physiologically active substance form a complex with said hydrophilic polymer-polylactide copolymer in the temperature range of 4° C. to 10° C. below the temperature at which the copolymer exhibits temperature-sensitivity.

12. A pharmaceutical composition for sustained-release of a substance comprising a temperature-sensitive carrier according to claim 1 and a physiologically active substance encapsulated within the carrier.

13. A pharmaceutical composition for sustained-release of a substance comprising a temperature-sensitive carrier according to claim 2 and a physiologically active substance encapsulated within the carrier.

14. A pharmaceutical composition for sustained-release of a substance comprising a temperature-sensitive carrier according to claim 3 and a physiologically active substance encapsulated within the carrier.

15. A pharmaceutical composition for sustained-release of a substance comprising a temperature-sensitive carrier according to claim 4 and a physiologically active substance encapsulated within the carrier.

16. A pharmaceutical composition for sustained-release of a substance comprising a temperature-sensitive carrier according to claim 5 and a physiologically active substance encapsulated within the carrier.

17. A pharmaceutical composition for sustained-release of a substance comprising a temperature-sensitive carrier according to claim 6 and a physiologically active substance encapsulated within the carrier.
